(19) United States
(12) Plant Patent Application Publication
(10) Pub. No.: US 2002/0184687 P1
(43) Pub. Date: Dec. 5, 2002

(54) Chrysanthemum plant named 'Gedi One Ylsap'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,858

(22) Filed: Jun. 4, 2001

(51) Int. Cl. A01H 5/00

(52) U.S. Cl. PLT/295

(57) ABSTRACT

A new and distinct Chrysanthemum plant variety characterized by a decorative type inflorescence, consistent flowering response to short days, blooming consistently after 49 days of short day length, free branching habit, yellow ray florets which resist fading with age, and a very uniform round growth habit.

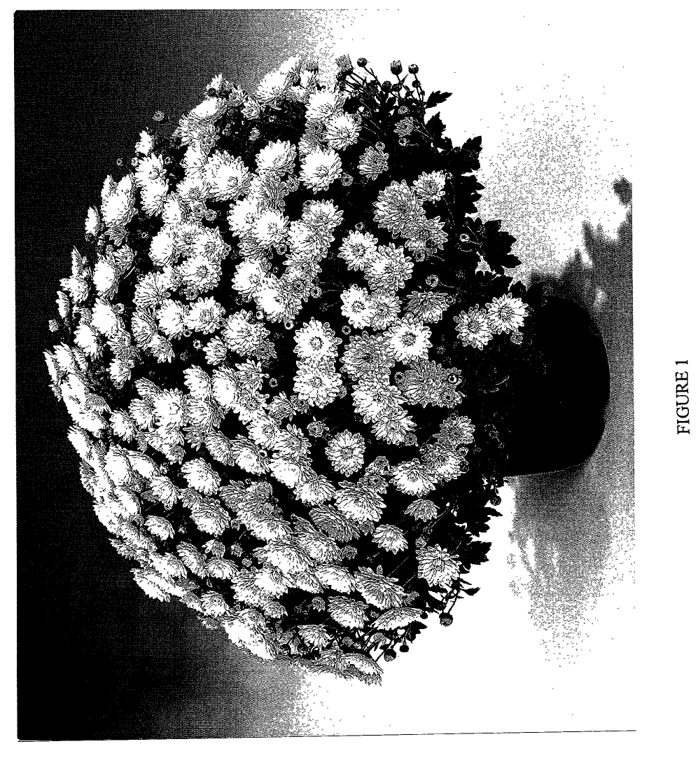

### BACKGROUND OF THE INVENTION

The present invention relates to a new and distinct cultivar of Chrysanthemum plant, botanically known as Dendranthema morifolium and herein after referred to by the cultivar name ‘GEDI ONE YLSAP.’

This new cultivar was discovered in the inventor's field in Belgium in October 1997. The new cultivar was discovered as a naturally occurring mutation on a single branch of the parent variety, ‘Sapiro,’ which is unpatented in the United States.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE YLSAP’ has not been observed under all possible environmental conditions. The phenotype may vary somewhat with variations in environment such as temperature, daylength, and light intensity, without, however, any variance in genotype.

The following traits have been repeatedly observe and are determined to be the unique characteristics of ‘GEDI ONE YLSAP.’ These characteristics in combination distinguish ‘GEDI ONE YLSAP’ as a new and distinct Chrysanthemum: 1. Decorative type inflorescence; 2. Consistent flowering response to short days, blooming consistently after 49 days of short day length; 3. Free branching habit; 4. Yellow ray florets, resisting fading with age; and 5. Very uniform round growth habit.

Plants of the new cultivar are similar to plants of the parent cultivar, ‘Sapiro,’ in most horticultural characteristics, however plants of the new cultivar differed from plants of the cultivar ‘Sapiro’ in the number of blooms and the color of the ray florets. The new cultivar has slightly less blooms, as well as different colored ray florets, compared to the parent variety.

The new cultivar can be compared to the Chrysanthemum cultivar ‘Dark Veria’ (disclosed in U.S. Plant Pat. No. 10,215). However plants of the new Chrysanthemum differed from the plants of the cultivar ‘Dark Veria’ in the following characteristics: 1. New cultivar naturally blooms approximately 1 week later than ‘Dark Veria;’2. New cultivar is taller and wider than ‘Dark Veria;’3. New cultivar has a more round plant shape and greater branching than ‘Dark Veria;’ and 4. New cultivar has more blooms per flowering branch ‘Dark Veria.’

### BRIEF DESCRIPTION OF THE PHOTOGRAPHS

The photograph shows a plant of ‘GEDI ONE YLSAP’ grown in a 2 gallon container. One cutting was used in the pot, planted mid May and grown under outdoor field conditions.

### DETAILED BOTANICAL DESCRIPTION

In the following description, color references are made to the Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE YLSAP’Commercial classification: Garden-type Chrysanthemum Parentage: Naturally occurring mutation on a single branch of the variety “Sapiro

PROPAGATION

Time to Rooting.—7 to 14 days at approximately 21° C.

Rooting habit.—Fine, fibrous

PLANT

Growth Habit.—Mounding herbaceous perennial

Height.—Approximately 24 cm

Growth rate.—Moderate

Branching Characteristics.—Free Branching

Length of Lateral Branches.—Approximately 21 cm

Number of leaves per lateral branch.—about 24

Stem color.—Near 138A

FOLIAGE

Leaf.—Arrangement: Alternate single Average Length: Approximately 6.2 cm Average Width: Approximately 3.6 cm Shape of blade: Ovate Apex: Acuminate Base: Attenuate Attachment: Stalked Margin: Serrate Texture of top surface: Lightly pubescent Texture of bottom surface: Lightly pubescent Color:

young foliage upper side: Near 137A youngfoliage under side: Near 137B mature foliage upper side: Near 137A maturefoliage underside: Near 137B venation upper side: Near 138C venation under side: Near 137C

Petiole.—Average Length: Approximately 2.1 cm

BLOOM

Inflorescence.—Flowering habit: Induced by darkness period greater than 13.5 hours, approximately 49 days of appropriate day length required to induce and develop blooms Inflorescenceform: Decorative Naturalflowering season: Approximately the first two weeks of October Number of inflorescences per stem: Approximately 15 Inflorescence diameter: Approximately 5.2 cm Inflorescence depth: 2.0 cm Inflorescence longevity on plant: Approximately 22-26 days Persistence: Persistent

Ray Florets.—Appearance: Matte Texture: Smooth Average Number perflower: 200 Shape: Oblanceolate Margin: Entire Apex: Obtuse Length: Approximately 3.1 cm Width: Approximately 0.8 cm Color: upper surface at first opening: Near 15C upper surface at maturity: Near 17D upper surface atfading: Near 15D under surface atfirst opening: Near 15B under surface at maturity: Near 17D under surface at fading: Near 15D

Disc florets.—Appearance: Shiny Texture: Smooth Average numberperflower: Approximately 60 Shape: Cylindric Apex: Obtuse Average Length: Approximately 1 cm Average Width: Approximately 0.2 cm Color: at first opening: Near 2A at maturity: Near 21A at fading: Near 17D

Peduncle.—Length: at terminal end (shortest): Approximately 5 cm at lateral end (longest): Approximately 9 cm Strength: Moderate Color: Near 137C Habit: Erect

Inflorescence bud.—Length: Approximately 1 cm Diameter: Approximately 1 cm Form: Globular Color: Near 15A Sepals: Appearance: Matte Texture: Lightly pubescent Number: Approximately 40 to 50 Shape: Ovate Margin: Entire Apex: Acute Color: upper side: Near 137A underside: Near 137A

REPRODUCTIVE ORGANS

Ray Florets.—Pistillate: Number ofpistils perflower: 1 Stigma shape: 2 branched Stigma color: Near 15B Style color: Near 15D Style length: 0.6 cm

Disc Florets.—Pistillate: Number ofpistils per flower: 1 Stigma shape: Cylindric Style length: 0.5 cm Staminate: Number ofstamens per flower: Approximately 5 Anther shape: Tubular Anther color: Near 15A Pollen color: Near 16B

OTHER CHARACTERISTICS Disease Resistance.—Neither resistance nor susceptibility observed

What is claimed is:

1. A new and distinct variety of Chrysanthemum plant named ‘GEDI ONE YLSAP’ as herein illustrated and described.
